(19) United States
(12) Patent Application Publication
(10) Pub. No.: US 2010/0004576 A1
(43) Pub. Date: Jan. 7, 2010

(54) Finger Splint Device for Preventing Contractures and the Like and Method of Using Same

(76) Inventor: Kathleen Molnar, Gilberton, PA (US)

Correspondence Address: Matthew T. Rogers, 15 South Franklin Street, Wilkes-Barre, PA, 18711-0075, US

(21) Appl. No.: 12/496,698

(22) Filed: Jul. 2, 2009

Related U.S. Application Data
(60) Provisional application No. 61/134,022, filed on Jul. 3, 2008.

(51) Int. Cl. A61F 5/00

(52) U.S. Cl. 602/22

(57) ABSTRACT

A finger splint device, particularly a finger splint device used to treat and relieve muscle contractures occurring in one or more fingers, comprises a shaped brace and a securing means for securing the finger splint device to the hand of the person wearing it. When wearing the finger splint device, no portion of the device covers the person's palm. The person is therefore able to have direct contact between his or her palm and the object the person desires to touch with his or her palm.

### CROSS-REFERENCE TO RELATED APPLICATIONS

This application claims priority under 35 U.S.C. §119(e)(1) from U.S. Provisional Patent Application No. 61/134,022, filed on Jul. 3, 2008, for “Finger Splint for Preventing Contractures and the Like,” by Kathleen Molnar, the disclosure of which is incorporated herein by reference.

### STATEMENT REGARDING FEDERALLY SPONSORED RESEARCH

Not Applicable.

### BACKGROUND

1. Field of the Invention

The invention relates to the field of medical devices used to immobilize parts of the human body. In particular, the invention relates to hand splints designed to treat and relieve the contracture of one or more muscles in a finger(s) following injury or surgery, or that occur as a result of a chronic condition such as arthritis.

2. Description of the Related Art

Medical devices designed to immobilize one or more of a person's fingers are well known. Simple splints comprising a flat piece of wood placed under a finger and secured thereto with tape have been in use for many years. Since then, more complex splints have been developed to accomplish specific treatment of a patient's injury or chronic condition involving his or her finger(s). For example, U.S. Pat. No. 5,947,915 (hereinafter, the “'915 Patent”) to Thibodo discloses a splint system comprising a channeled splint member body and fastening straps. U.S. Pat. No. 6,110,136 to Belkin discloses a digit splint device comprising an angularly inclined base and an ovoid extending upward from the base. U.S. Pat. No. 7,001,352 to Farrell et al. (hereinafter, the “'352 Patent”) discloses a dynamic splint comprising a support, fingertip caps, and a plurality of adjustable leads. U.S. Pat. No. 6,860,863 to Chow discloses a carpal tunnel splint comprising a first and second sleeve. U.S. Pat. No. 6,371,932 to Foote (hereinafter, the “'932 Patent”) discloses a finger attitude correction device. U.S. Pat. No. 6,592,584 to Agee et al. (hereinafter the “'584 Patent”) discloses a device for correcting finger joint contractures.

The are a number of medical reasons for why a patient needs to immobilize parts of his or her hand, including the fingers. For example, a patient with a fractured finger will often have the fractured finger immobilized to allow the natural process of bone healing to occur. Alternatively, patients may suffer muscle contractures in one of their fingers which causes the finger to become stuck in a contracted position (i.e., the patient's finger involuntarily contracts into a “claw like” position). Muscle contractures can be caused by injury, surgery, or by a chronic condition such as arthritis. One treatment option for the affliction of a muscle contracture of a finger is to stretch the contracted finger. Stretching causes the contracted muscles in the finger to relax and release the finger from its “claw like” position. Stretching may be achieved by physically stretching the affected finger and manually holding it in the stretched position. While effective, the patient holding his or her finger in the stretched position, or alternatively the physical therapist holding the patient's finger in the stretched position, will eventually tire. The solution to this problem is the use of a splint device that holds the finger in the stretched position for a extended period of time.

The problems with known splint devices designed to immobilize one or more fingers of the human hand are (i) they require a significant amount of dexterity to put on and (ii) all or a portion of the splint device covers the patient's palm. While young, healthy patients may have little difficulty putting on a known splint device, elderly and disabled patients, especially those suffering from arthritis, will struggle to put on splint devices such as the one disclosed in the '932 Patent and the one disclosed in the '352 Patent. They will most likely require the help of an assistant, who may or may not be available at the time the patient needs to wear the splint device.

As mentioned above, known splint devices, such as the devices disclosed in the '915 Patent and the '584 Patent, cover the palm of the patient's hand. While this may not create a significant problem for young, healthy patients, elderly patients, due to the fragile condition of their skin, commonly suffer skin “breakdowns” (e.g., sores and blisters) whenever moisture (e.g., in the form of sweat) collects in the narrow area between the patient's skin and the splint device. Moreover, a number of elderly patients use mobility assistance devices such as canes and walkers and the inability of an elderly patient to make direct skin contact with such mobility assistance devices makes it more difficult for some patient to use such devices.

There is therefore a need for a finger splint device that sufficiently immobilizes a patient's finger(s) to treat injuries and contractures, is easy to put on, and does not cover a patient's palm.

### SUMMARY

The present invention is directed to a novel finger splint device, and a method of using same, that satisfies the need for an effective, easy to put on medical device that immobilizes one or more fingers of a patient's hand yet allows an elderly or disabled patient to better use his or her hand while avoiding common skin-related ailments associated with known splint devices. Putting on a finger splint device having features of the present invention involves simply threading the affected finger through an opening in the device and securing the device to the hand of the person wearing it. When wearing a finger splint device having features of the present invention, the elderly or disabled patient is able to have direct contact between his or her palm and the object the patient desires to touch with his or her palm. Moreover, the patient's palm remains uncovered while wearing a finger splint device having features of the present invention which prevents the occurrence of skin “breakdowns” commonly experienced when a medial device, or any portion thereof, covers the patient's palm. A finger splint having features of the present invention comprises a shaped brace, shaped to fit under and around one or more fingers and over the hardel of the patient's hand, and a means for securing the shaped brace to the patient's hand.

### BRIEF DESCRIPTION OF DRAWINGS

FIG. 1 is a perspective view of a finger splint device embodying features of the present invention.

FIG. 2 is a bottom plan view of a finger splint device embodying features of the present invention.

FIG. 3 is a top plan view of a finger splint device embodying features of the present invention.

FIG. 4 is a side view of a finger splint device embodying features of the present invention.

### DETAILED DESCRIPTION

In the Summary above, and in the Detailed Description and the Claims below, reference is made to particular features of the invention. It is to be understood that the disclosure of the invention in this specification includes all appropriate combinations of such particular features. For example, where a particular feature is disclosed in the context of a particular embodiment or a particular claim, that feature can also be used, to the extent appropriate, in the context of other particular embodiments and claims, and in the invention generally.

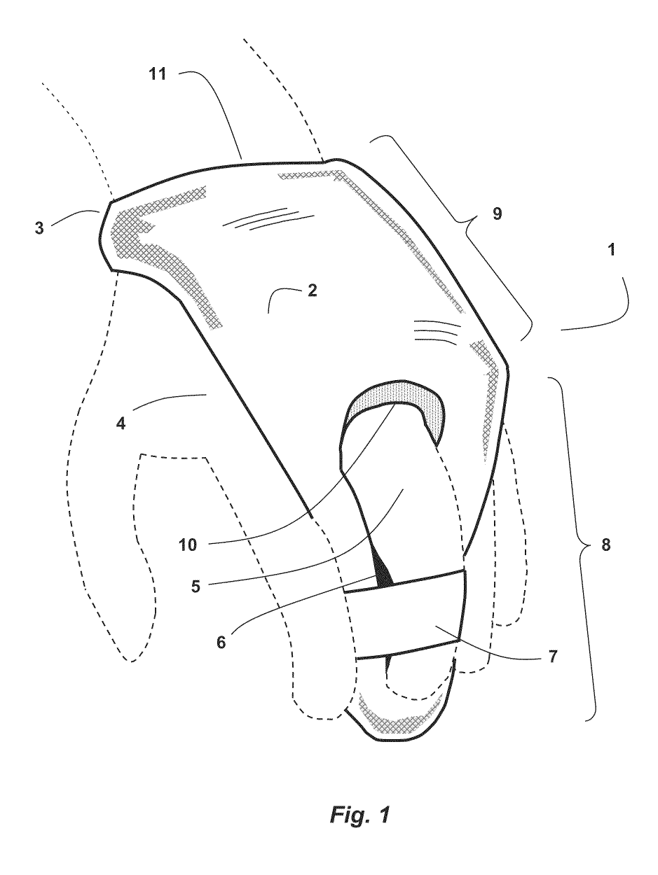

As shown in FIG. 1, a finger splint device 1 comprises a shaped brace 2 that is shaped to fit underneath and around one or more fingers of a patient's hand 4 and a means 3 for securing the shaped brace 2 to the patient's hand 4. The shaped brace 2 comprises a distal portion 8 and a proximal portion 9. The distal portion 8 and the proximal portion 9 are permanently connected to each other, preferably seamlessly. The distal portion 9 is shaped to fit underneath and around the patient's immobilized finger 5 as shown in FIG. 1. The proximal portion 9 is shaped to fit above the hardel 11 of the patient's hand 4 as shown in FIG. 1. In the region of the shaped brace 2 where the distal portion 8 and the proximal portion 9 meet, there is an opening 10. The opening 10 can be of any shape and size, as long as it is big enough to allow the patient's immobilized finger 5 to thread through the opening 10. The shaped brace 2 can be made of any solid material, and is preferably made of medical grade foam. Alternatively, the shaped brace 2 may be made out of plastic, fabric, an air-filled bladder (see, e.g., the air-filler bladder device disclosed in U.S. Pat. No. 5,125,400 to Johnson), or a gel-filled bladder.

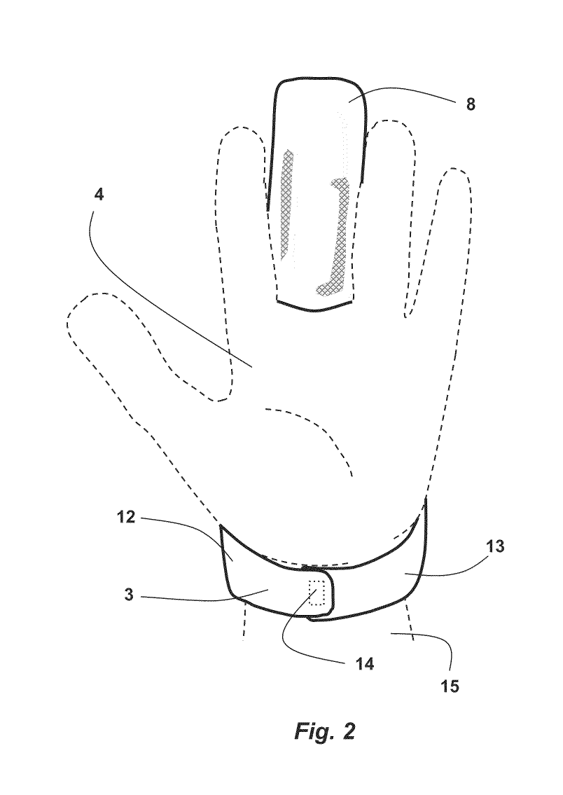

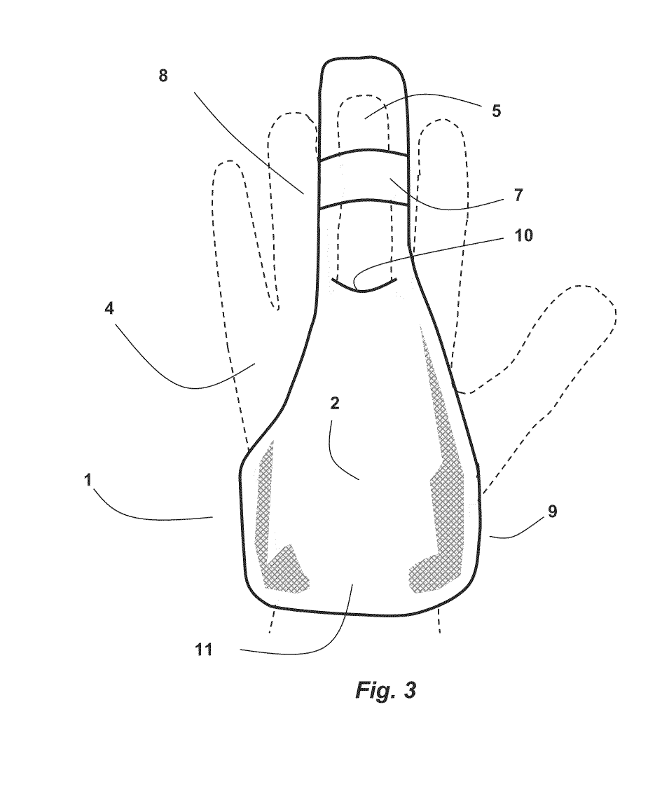

As shown in FIGS. 2 and 3, the shaped brace 2 comprises a securing means 3 for securing the shaped brace 2 to the patient's hand 4. Any securing means 3 can be used, provided that the shaped brace 2 is restrained from falling off the patient's hand 4 when the finger splint device 1 is in use. In the preferred embodiment shown in FIG. 2, the securing means 3 comprises a first strap 12 and a second strap 13 that are affixed together via Velcro®-like hook and pile strips 14. Alternatively, the securing means 3 may comprise a known elastic bandage that is wrapped around the end of the proximal portion 9 of the shaped brace 2 and the patient's arm 15, and is secured in place using known elastic bandage fasteners (e.g., aluminum clips).

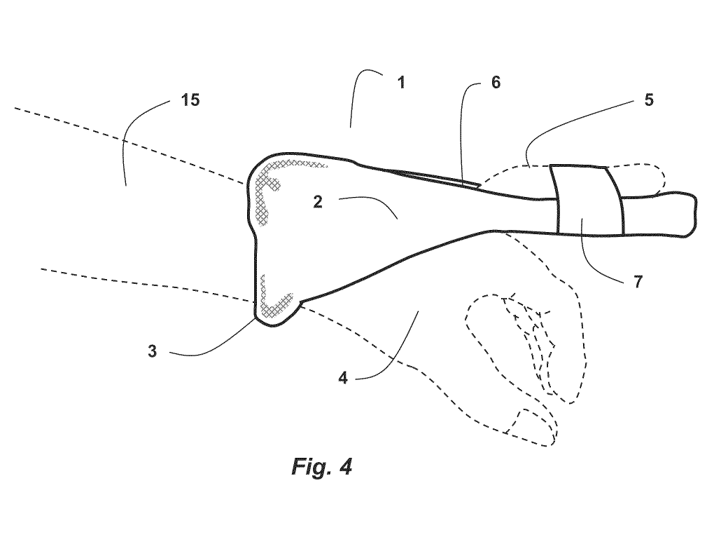

A preferred embodiment of a finger splint device 1 embodying features of the present invention further comprises a channel 6 in the distal portion 8 of the shaped brace 2, such as the one shown in FIGS. 1 and 4. As shown in FIGS. 1 and 4, the channel 6 is shaped to surround the bottom half of the circumference of the patient's immobilized finger 5. The channel 6 allows for the patient's immobilized finger 5 to be further secured against the distal portion 8 of the shaped brace 2.

A preferred embodiment of a finger splint device 1 embodying features of the present invention further comprises a finger strap 7. As is shown in FIG. 1, the finger strap 7 is wrapped around the patient immobilized finger 5 and the distal portion 8 of the shaped brace 2. Alternatively, the finger strap 7 can be permanently attached to the distal portion 8 of the shaped brace 2 in such a way as to ensure the finger strap 7 surrounds the top half of the circumference of the patient's immobilized finger 5. The finger strap 7 can be made of any material, and is preferably made of an elastic material such as, e.g., a knitted elastic bandage. The finger strap 7 allows for the patient's immobilized finger 5 to be further secured against the distal portion 8 of the shaped brace 2.

In the preferred embodiment shown in FIGS. 1 through 4, the method for treating muscle contractures in one or more fingers comprises the steps of: (a) threading the finger 7 suffering the contracture through the opening 10 of the shaped brace 2; (b) positioning said finger 7 flat, or relatively flat, against said distal portion 8 of said shaped braced 2 (preferably in the channel 6), and (c) securing said finger splint device 1 to the hand 4 of the person wearing said finger splint device 1 via the securing means 3. In addition, the method could further comprise the step of threading the patient's immobilized finger 7 through the finger strap 12 and tightening the finger strap 12 to secure the finger 5 to the distal portion 8 of the shaped brace 2, and preferably within the channel 6.

What is claimed is:

1. A finger splint device, comprising:
(a) a shaped brace, said shaped brace comprising (i) a distal portion shaped to fit underneath and around one or more fingers of a patient's hand, (ii) a proximal portion shaped to fit on top of the hardel of said hand, and (iii) an opening in the area of the shaped brace where said distal portion and said proximal portion meet, said opening being large enough to allow a finger to be threaded through; and
(b) a securing means for securing said shaped brace to said hand.

2. The finger splint device of claim 1, wherein said shaped brace is made of medical grade foam.

3. The finger splint device of claim 1, wherein said shaped brace is made of plastic.

4. The finger splint device of claim 1, wherein said shaped brace is made of fabric.

5. The finger splint device of claim 1, wherein said shaped brace is made of an air-filled bladder.

6. The finger splint device of claim 1, wherein said shaped brace is made of a gel-filled bladder.

7. The finger splint device of claim 1, wherein said securing means comprises opposing straps attached to said proximal portion of said shaped brace and affixed together.

8. The finger splint device of claim 1, wherein said securing means comprises an elastic bandage wrapped around said proximal portion of said shaped brace and the wrist of the person wearing said finger splint device.

9. The finger splint device of claim 1, wherein said shaped brace further comprises a channel molded into said distal portion of said shaped brace.

10. The finger splint device of claim 1, wherein said shaped brace further comprises a finger strap.

11. The finger splint device of claim 10, wherein said finger strap is made of an elastic material.

12. The finger splint device of claim 8, wherein said finger strap is made of a knitted elastic bandage.

13. A method for treating muscle contractures in one or more fingers comprising the steps of:
(a) threading the finger(s) suffering the contracture through the opening of a finger splint device, said finger splint device comprising a shaped brace having a distal portion and a proximal portion with said opening located where said distal portion and said proximal portion meet and a securing means for securing said shaped brace to the hand of the person wearing said finger splint device; and
(b) positioning said finger(s) flat, or relatively flat, against said distal portion of said shaped braced, and
(c) securing said finger splint device to said hand.

14. The method of claim 13, further comprising the step of threading said finger(s) through a finger strap, said finger strap located on the distal portion of said shaped brace.
